(19) United States
(12) Patent Application Publication
(10) Pub. No.: US 2016/0007815 A1
(43) Pub. Date: Jan. 14, 2016

(54) FLOOR SUCTION DEVICE

(71) Applicant: BIG FOOT SUCTION, LLC, Atlanta, GA (US)

(72) Inventor: Nicholas A. ESPOSITO, Canton, GA (US)

(73) Assignee: BIG FOOT SUCTION, LLC, Atlanta, GA (US)

(21) Appl. No.: 14/859,536

(22) Filed: Sep. 21, 2015

Related U.S. Application Data
(63) Continuation-in-part of application No. 14/719,705, filed on May 22, 2015.
(60) Provisional application No. 62/003,347, filed on May 27, 2014.

(51) Int. Cl. A47L 7/00; A61B 19/00

(52) U.S. Cl. CPC A47L 7/0004; A61B 19/00; A47L 7/0009; A61B 2019/48

(57) ABSTRACT

A surgical floor suction device includes a hollow shell defining a central aperture and an annular internal suction chamber surrounding the central aperture. The shell has a suction surface with a plurality of suction holes spaced around the central aperture and extending through the shell into fluid communication with the internal suction. The suction device further includes a suction coupling extending from the hollow shell and defining a suction conduit extending into fluid communication with the internal suction chamber.

### CROSS-REFERENCE TO RELATED APPLICATIONS

This application is a continuation-in-part of U.S. Non-Provisional Patent application Ser. No. 14/719,705 filed May 22, 2015, which claims the benefit of U.S. Provisional Patent Application Ser. No. 62/003,347 filed May 27, 2014, the entireties of which are hereby incorporated herein by reference for all purposes.

### TECHNICAL FIELD

The present invention relates generally to the fields of medical and surgical equipment and fluid collection devices, and more particularly to a mobile suction device for removing liquids from a floor or other surface in hospital operating rooms, surgical suites, medical offices and/or other environments.

### BACKGROUND

During surgery or other medical procedures it is common for fluids to collect on the floor and or other surfaces of an operating room, surgical suite, medical or dental office or other areas. Often, towels, blankets, surgical matting or other absorbent materials may be used to collect the fluid. Collecting surgical fluids by hand can be messy, and potentially exposes personnel to contaminants. Alternatively, a suction disc may be provided to collect fluid from the floor surface. But suction discs may be expensive, require additional cleaning and maintenance after use, and typically are stationary on the floor surface. Accordingly, it can be seen that needs exist for an improved surgical floor suction device. It is to the provision of a surgical floor suction device meeting these and other needs that the present invention is primarily directed.

### SUMMARY

In example forms, the present invention provides a portable and disposable surgical floor suction device for absorbing and removing unwanted fluids from a floor surface. In representative embodiments, the invention provides a movable, renewable and durable suction and absorption apparatus dedicated to sucking and absorbing fluids off of the floor during and after surgical cases. It is easily repositioned with the use of a person's foot. The movability, renewability, and multi-functional capabilities of the product's utility and design provide significant advantage over previously known methods and equipment.

In one aspect, the present invention relates to a surgical floor suction device including a movable absorptive mat assembly, a first conduit, and a second conduit. The mat assembly includes an absorption material and a sliding netting material. The first conduit includes a loop of tubing having at least one fluid collection opening formed therein and a Y-connector. The second conduit is provided for connection between the Y-connector of the first conduit and a suction system.

In another aspect, the invention relates to a surgical floor suction device including a mat assembly having a pocket, a first conduit segment for fitting within the pocket, and a second conduit segment having a first end attached to or extending from the first conduit segment and a second end for removably coupling to a suction device.

In still another aspect, the invention relates to a movable surgical floor suction device for collecting fluids from a floor surface including a mat assembly, and at least one suction conduit. The mat assembly includes an absorption material layer and a screen material layer. The screen material layer is affixed to a bottom portion of the absorption material and configured for low-friction sliding along the floor surface, and wherein affixing the screen material layer to the absorption material layer defines a pocket. At least a portion of the suction conduit is positioned within the pocket and includes a length of tubing having at least one opening formed therein for suctioning fluid from the mat assembly, and a distal end for coupling to a suction device. Preferably, the device is configured such that a human user's foot or shoe sole can be placed atop the mat assembly to move the mat assembly across the floor surface to collect unwanted fluids therefrom.

In yet another aspect, the present invention relates to a method of removing unwanted fluids from a floor surface. The method includes providing a surgical floor suction device, the surgical floor suction device including a mat assembly, a first conduit and a second conduit, the mat assembly including an absorption material and a netting material, the netting material being affixed to a bottom portion thereof; placing the surgical floor suction device on the floor surface wherein the netting material is in contact with the floor surface; placing a foot atop the mat assembly; and moving the mat assembly across the floor surface by movement of the foot to collect the unwanted fluids.

In another aspect, the invention relates to a surgical floor suction device including a hollow shell defining an internal suction chamber and a suction surface having a plurality of suction holes extending therethrough in fluid communication with the internal suction chamber. The floor suction device preferably further includes a suction coupling extending from the hollow shell and defining a suction conduit extending into fluid communication with the internal suction chamber.

In another aspect, the invention relates to a surgical floor suction device including a hollow shell defining a central aperture and an annular internal suction chamber surrounding the central aperture. The shell preferably has a suction surface with a plurality of suction holes spaced around the central aperture and extending through the shell into fluid communication with the internal suction chamber.

In another aspect, the invention relates to a surgical floor suction device including a hollow shell defining a central aperture and an annular internal suction chamber surrounding the central aperture. The shell preferably has a suction surface including a plurality of suction holes spaced around the central aperture and extending through the shell into fluid communication with the internal suction. The device preferably also includes a suction coupling extending from the hollow shell and defining a suction conduit extending into fluid communication with the internal suction chamber.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a top view of a surgical floor suction device according to an example embodiment of the present invention.

FIG. 2 is a bottom view of the surgical floor suction device of FIG. 1.

FIG. 3 is a perspective view of a first conduit segment of the surgical floor suction device of FIG. 1.

FIG. 4 is a detailed view of a portion of the first conduit of FIG. 3, showing a portion thereof comprising a plurality of openings.

FIG. 5 shows the internal conduit segment arrangement of another embodiment of a floor suction device according to an example form of the invention.

FIG. 6 shows a top view of another embodiment of a floor suction device according to an example form of the invention.

FIG. 7 shows a bottom view of another embodiment of a floor suction device according to an example form of the invention.

FIG. 8 shows a hose-only embodiment of a floor suction device according to an example form of the invention.

FIG. 9 shows a person using a floor suction device to clean up a fluid spill in accordance with an embodiment of the present invention.

FIG. 10 is a perspective view of a surgical floor suction device according to another example embodiment of the present invention.

FIG. 11 is an assembly view of the surgical floor suction device of FIG. 10.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

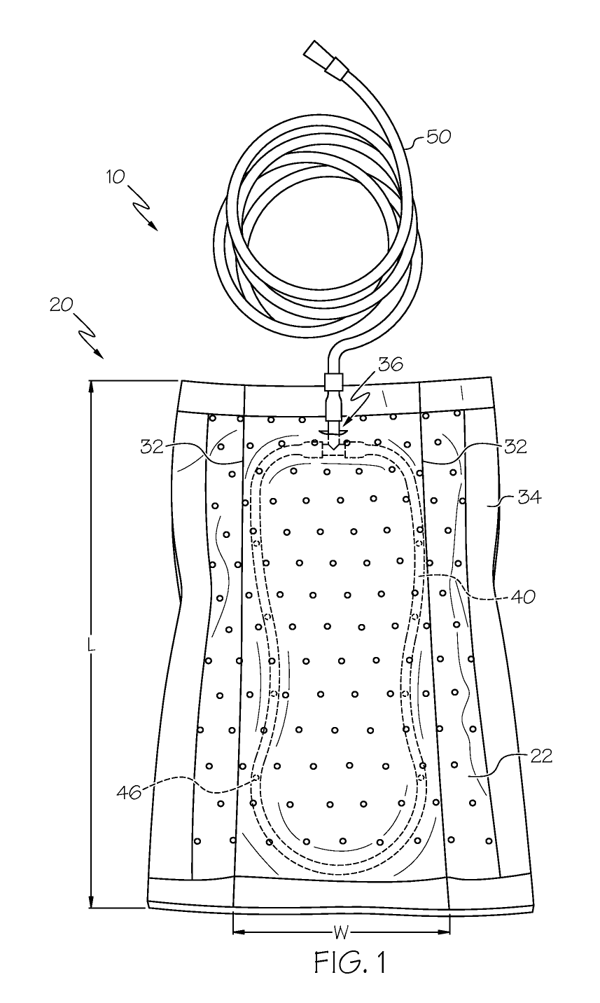

With reference now to the drawing figures, wherein like reference numbers represent corresponding parts throughout the several views, FIG. 1 shows a floor suction device 10 according to an example embodiment of the present invention. As depicted, the device 10 generally comprises a mat assembly 20, a first conduit 40, and a second conduit 50. The first conduit 40 is generally contained within the mat assembly 20, and the second conduit 50 is connected to a suction device such as a surgical suction pump, and communicates with the first conduit 40. In alternate embodiments, the first and second conduits form segments of a single continuous and unitary length of conduit, with a first segment of the conduit within the mat assembly and a second segment of the conduit extending outside of the mat assembly. Preferably, the floor suction device 10 is capable of removing unwanted fluid from a floor surface by wicking and absorption of the fluid into the mat assembly 20 and sucking the fluid from the mat assembly into the first conduit 40 and out through the second conduit for collection and disposal by a suction device. Preferably, the floor suction device 10 is capable of being moved on the floor surface by a user's foot, for example wherein the foot or shoe sole is to be placed generally on the top surface of the mat assembly 20. Alternately, the floor suction device 10 can remain stationary on the floor surface.

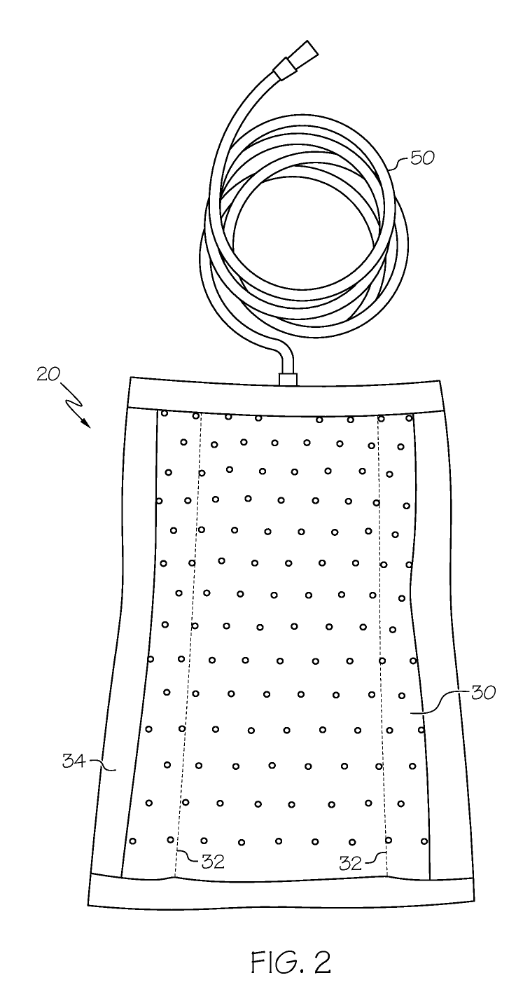

As depicted in FIGS. 1-2, the mat assembly 20 is generally rectangular in shape and is formed from an absorption mat 22 and a screen-like mesh or netting material 30. Optionally, the mat assembly 20 can be shaped as desired, for example, circular, polygonal, oval, etc. In one form, the mat assembly 20 is constructed such that the netting material 30 is coupled to at least a portion of the bottom of the absorption mat 22, which can be affixed thereto by stitching, adhesives, tape, heat bonding, etc. The mesh 30 may comprise a natural or synthetic rubber, plastic or other material(s), and is preferably non-absorbent and provides a low coefficient of friction for sliding along common flooring surfaces. Preferably, the absorption mat 22 comprises two or more sheets of matting material having a high absorbency and wicking properties, which are generally aligned along their edges and affixed together. Alternately, the absorption mat 22 may comprise a single sheet of matting material that is generally folded to form two layered sheets.

In example forms, laterally offset stitches 32 are provided along the length of the mat assembly 20 to affix the two sheets of matting material together and to affix the absorption mat 22 to the netting material 30. By affixing the matting material of the mat 22 and the netting material 30 together by stitches 32, a generally centrally positioned pocket, slot, or reservoir is formed within the mat 22 (e.g., between the two sheets of matting material) to contain the first conduit 40 therein. In one example form, the pocket comprises a length L and width W, which are configured to provide an area wherein a user's foot or shoe sole can be placed thereon (e.g., the foot being placed on a top portion of the mat assembly), for example to move the device 10 on the floor surface to proactively remove fluid therefrom (as will be described below). Optionally, indicia can be provided on the pocket to indicate placement and position of the user's foot. Further optionally, one or more side edges of the assembly 20 can comprise affixing means 34 for additional support of the mat assembly 20, which may include gaffe tape, additional stitching, glue, adhesive, heat bonding, etc.

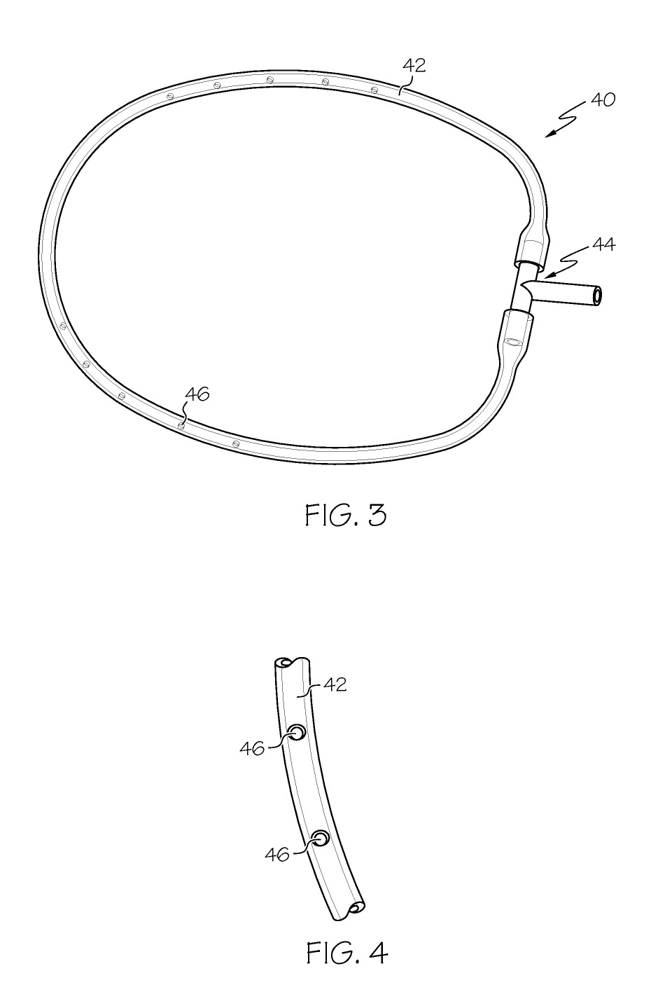

FIG. 3 shows the first conduit 40 in greater detail. Generally, the first conduit 40 comprises a length of tubing 42, a Y-shaped or T-shaped coupling or connector 44, and a plurality of openings 46 formed in the tubing 42. Preferably, the ends of the tubing 42 are connected to first and second oppositely opposed ends of the Y-shaped or T-shaped connector 44 to generally form a loop, and a third end of the connector 44 (generally extending perpendicular from the oppositely opposed ends) is further connected to an end fitting of the second conduit 50. In example forms, the tubing 42 is generally shaped to form a generally oval or elliptical loop corresponding to the shape of a human foot or shoe sole. In one form, the tubing is shaped and sized to adequately fit within the pocket of the mat assembly 20 and provide an area therein that is generally sized and shaped to receive the foot or shoe sole within or over the loop of tubing. Preferably, a slot or opening 36 is provided in an upper portion of the absorption mat 22 (allowing access to the pocket) such that the third end of the connector 44 can extend therethrough to be connected with the proximal end of the second conduit 50. The distal end of the second conduit 50 similarly comprises an end fitting to be connected to an external suction device such as a suction pump or blower and suction canister fluid collection system.

FIG. 4 shows a portion of the first conduit 40 in greater detail. Preferably, a plurality of openings 46 are spaced along the length of the tubing 42 such that fluid wicked by the mat 22 is drawn into the first conduit 40 and then suctioned out the second conduit 50 towards the suction device. In one form, the plurality of openings are provided along the lengthwise sides of the tubing, for example wherein about 5 openings 46 are provided along each side of the tubing 42 (see FIG. 1). In another form, the tubing 42 can comprise about 10 openings 46 along each side of the tubing 42 (e.g., total of about 20 openings). Optionally, fewer or more openings are provided along the tubing 42.

In use, the floor suction device 10 preferably provides a method for the removal of unwanted fluid from a floor surface. In example forms, the floor suction device 10 is preferably placed on the floor surface with the netting material 30 facing down and in contact with the floor surface in the area of fluid to be collected. A user's foot is then placed atop the centrally positioned pocket of the mat assembly 20 (and within the loop of first conduit 40, see foot image) to allow the user to move the mat assembly 20 across the floor surface. As the mat assembly 20 begins to contact the fluid, the fluid is wicked into the mat 22 and drawn under suction into the first conduit 40. The fluid is subsequently drawn under suction from the first conduit 40 into the second conduit 50 and towards the suction device. Preferably, the netting material 30 in contact with the floor surface allows the assembly 20 to move smoothly and without excess friction across the floor surface. Thus, despite any quantity of fluid being wicked within the absorption mat 22, the mat assembly 20 is preferably capable of freely sliding on the floor surface by providing the netting material 30 on the bottom surface of the mat assembly 20.

In example embodiments, the absorption mat 22 comprises one or more sheets of woven or non-woven surgical absorption matting that is generally sized to be about 10″×30″, which is folded in half such that the overall size is about 10″×15″. The netting material is formed from a rubber mesh-like screen that is generally sized to be about 10″×15″. Thus, in example forms, the mat assembly is generally rectangular-shaped and about 10″×15″. The pocket is generally sized to have a length L of about 15″ and a width W of about 6″. The first conduit 40 generally comprises a 1/4″ or 3/8″ surgical vinyl tubing having a length of about 30″. Preferably, the openings 46 are generally circular and are sized to have a diameter of about 1/16″. The Y-shaped connector generally comprises three ribbed ends that all are sized to removably engage tubing having a diameter of about 3/8″. The second conduit 50 generally comprises about 10′ of 1/4″ or 3/16″ suction tubing such as for example Medline sterile suction tubing. One or both ends of the second conduit 50 optionally comprise adaptor fittings to provide for connecting to the Y-shaped connector 44 and the suction device. The optional affixing means 34 can include gaffing tape having a width between about 1″-2″. The thread for forming the stitches 32 can be in the form of heavy duty polyester or other material. A footprint image, instructions, safety warnings, or other images, indicia or material may optionally be printed, painted, embossed, dyed, or otherwise applied to the mat 22. Optionally, additional/other glues, adhesives, dyes, paints, fabrics, tubes, connectors, etc. may be incorporated with the floor suction device 10 as desired. Preferably, the dimensions and specifications as recited herein are according to one example form of the present invention. Optionally, the dimensions, specifications, shapes, components, etc. as recited herein can be chosen as desired. For example, in additional example embodiments, the absorption mat 22 can be sized as desired. In one form, the mat 22 is sized to be about 20″×30″ (e.g., about double the area of the 10″×15″ mat), and in another form, the mat 22 is sized to be about 5″×7.5″ (e.g., about half the area of the 10″×15″ mat). In other example forms, the mat 22 can be about 10″×15″, about 15″×20″, about 10″×30″, or other sizes as desired. Preferably, the other components to be assembled with the absorption mat 22 (e.g., netting material, etc.) are sized accordingly. Furthermore, as described above, the quantity of openings 46 formed in the tubing 42 can be changed according to the size of the mat 22. In typical embodiments, the tubing 42 of the 10″×15″ mat comprises about 10 openings 46, the tubing 42 of the 20″×30″ mat comprises about 20 openings 46, and the tubing 42 of the 5″×7.5″ mat comprises about 10 openings 46.

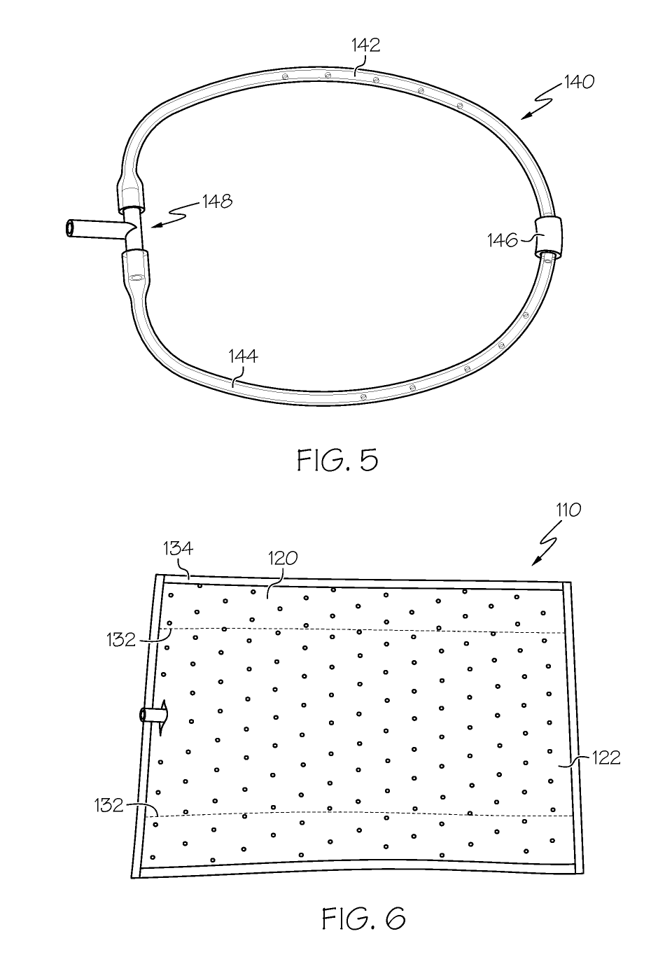

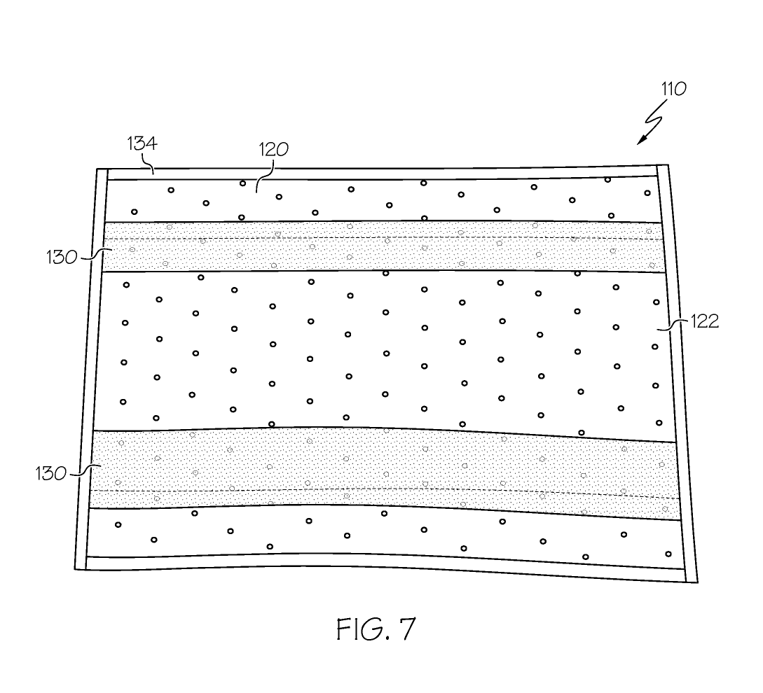

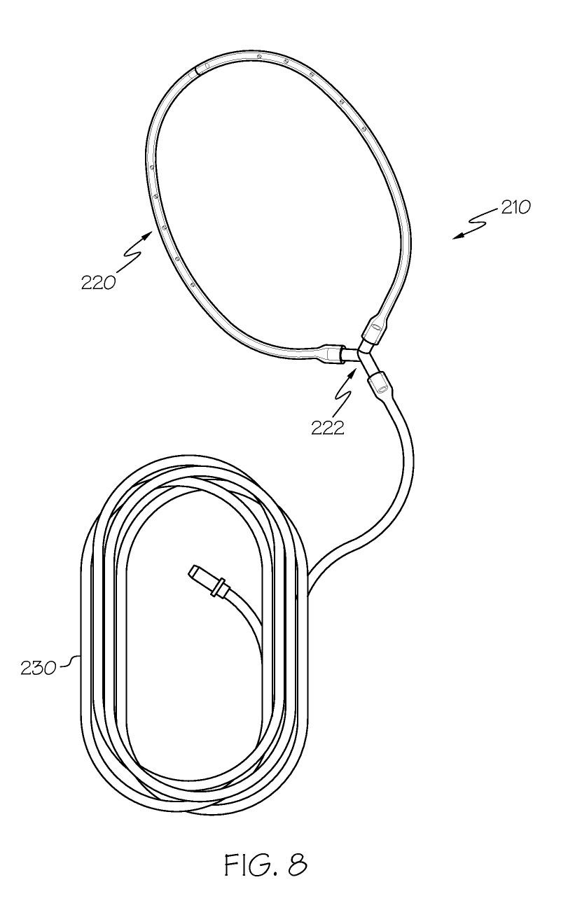

FIGS. 5-7 show another example embodiment of a floor suction device 110 according to the present invention. As depicted, the device 110 generally comprises a mat assembly 120, a first conduit segment 140, and a second conduit segment. The first conduit segment 140 comprises a loop of tubing defining a first section 142 and a second section 144, having a fluid-impermeable plug 146 at opposite the Y-coupling 148, segregating the loop into separate suction sections. One or more fluid suction holes are spaced along each of the sections 142, 144. For example in the depicted embodiment, five suction holes are spaced along each of the sections. The mesh 130 is applied in one or more strips to the bottom of the mat 120, for example extending lengthwise along the mat, covering a portion of the bottom of the mat and leaving a portion of the absorbent material 122 exposed along the bottom of the mat for contact with the floor. Seams 132 retain the mesh in place on the mat and define the pocket within which the first conduit segment 140 is positioned, with the foot applied generally centrally within the loop of the first conduit segment. One inch (1″) gaff tape 134 is applied in a border around the edges.

In an additional embodiment, one or more suction devices can be packaged in a kit. One example kit includes two suction devices, at least one length of second conduit tubing, and instructions for use, packaged within a poly bag or other packaging. In a further embodiment, the mat assembly, the first conduit, or both are disposable and/or replaceable.

FIG. 8 shows another example embodiment of a floor suction device 210 according to the present invention. As depicted, the device 210 generally comprises a first conduit segment 220 and a second conduit segment 230. The first conduit 220 can operate as a hose-only suction device without an absorbent mat and the second conduit 230 is connected to a suction device such as a surgical suction pump, and communicates with the first conduit 220. In this embodiment, the first conduit 220 and the second conduit 230 are lengths of flexible tubing joined by a T- or Y-coupling 222 with the first conduit segment forming a loop having a plurality of suction openings spaced along its length in similar fashion to that described above. In alternate embodiments, the first and second conduits form segments of a single continuous and unitary length of conduit, with a first segment of the conduit laid on the floor in the area of a possible spill and a second segment of the conduit extending away from the possible spill area. Preferably, the floor suction device 210 is capable of removing unwanted fluid from a floor surface by suction of the fluid from the floor into the first conduit 220 and out through the second conduit 230 for collection and disposal by a suction device. Preferably, the floor suction device 210 is capable of being moved on the floor surface by a user's foot. Alternately, the floor suction device 210 can remain stationary on the floor surface. In alternate embodiments, the hose-only suction device 210 may be provided in combination with a removable absorbent mat similar to the above-described embodiments, such that a user can selectively install or remove the mat and use the device as a hose-only device or a hose-and-mat device.

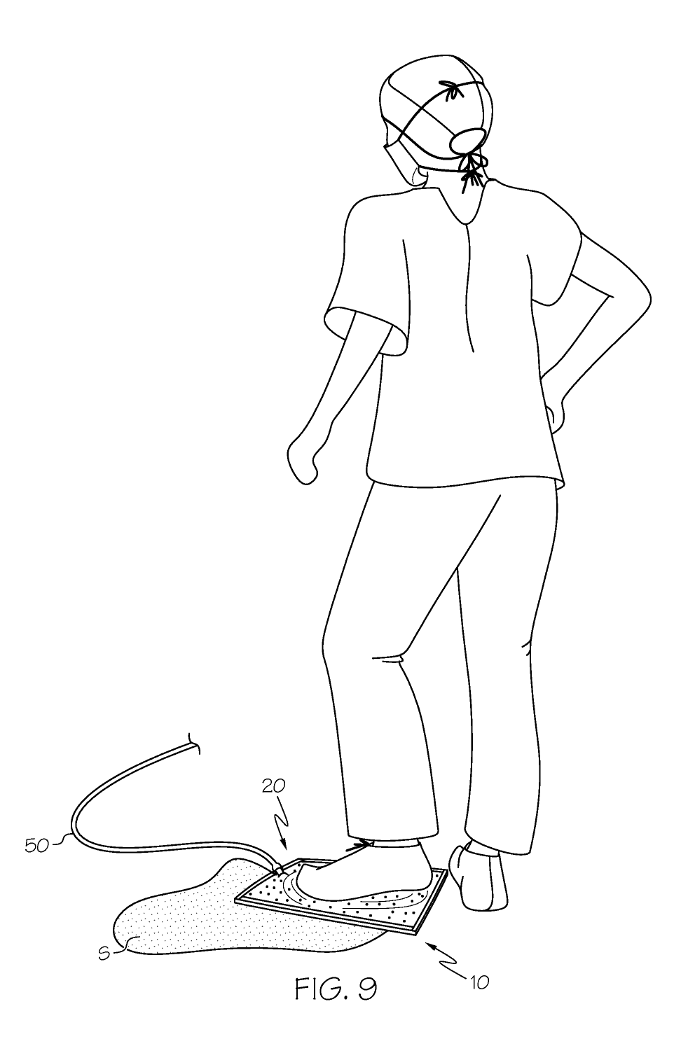

In additional example embodiments, the present invention comprises a method of removing fluids from a floor surface. The method preferably includes providing an absorbent suction device comprising a mat assembly, a first conduit segment and a second conduit segment, the mat assembly comprising an absorption material and a free-sliding mesh material, the mesh material being affixed to a bottom portion of the mat; placing the floor suction device on the floor surface wherein the netting material is in contact with the floor surface; placing a foot atop the mat assembly; and moving the mat assembly across the floor surface by movement of the foot to collect the fluids. FIG. 9 shows an exemplary embodiment of the present invention in which a person removes a fluid or spill S from a surface using the floor suction device 10 by placing the mat assembly 20 on a surface and moving the mat with one foot while second conduit 50 carries the fluid or spill away from the surface.

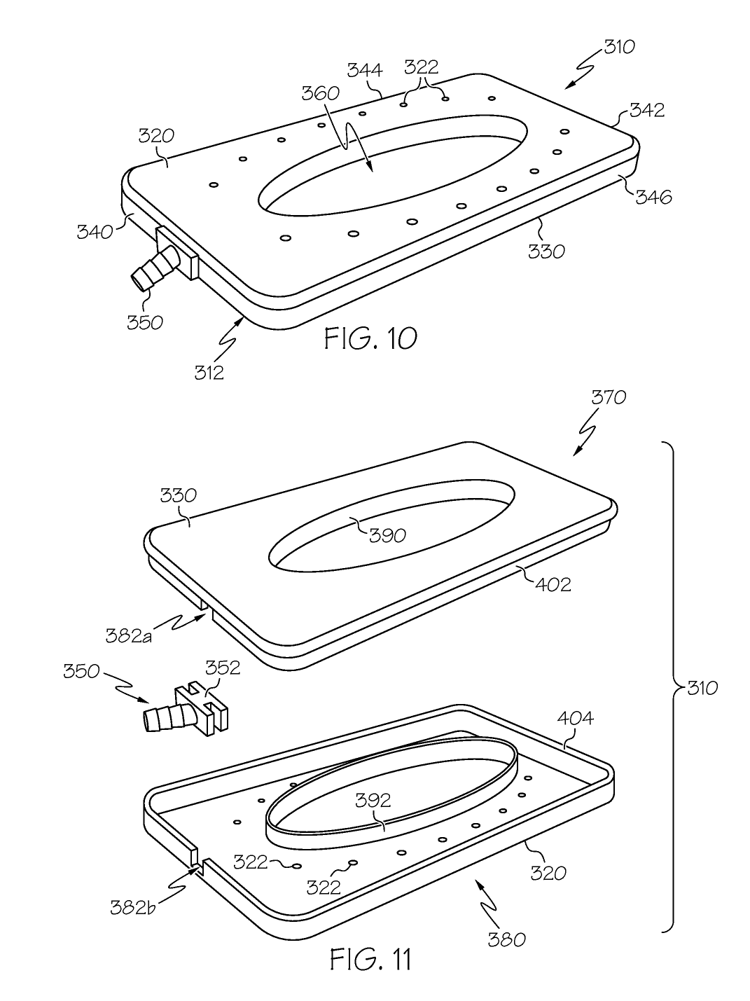

FIGS. 10 and 11 show another example embodiment of a surgical floor suction device 310 according to the present invention. The suction device 310 comprises a generally rectangular hollow shell or housing 312 having a bottom panel or suction surface 320, a top panel or user-interface surface 330, a front face 340, back face 342, and first and second side faces 344, 346. A ribbed tubing coupling 350 having a suction conduit or lumen therethrough extends from the front face 340 or other connection point of the housing 312. A generally oval or elliptical central aperture or window 360 extends through the housing through the top and bottom panels 330, 320. In example embodiments, the window 360 has a length of about 30%-75% of the overall length of the housing 312, and a width of about 30%-75% of the overall width of the housing. In this manner, the housing 312 defines a generally annular or ring-shaped internal suction chamber between the top and bottom panels 330, 320, with the central window defining the inner diameter of the ring-shaped suction chamber. The bottom panel or suction surface 320 defines a plurality of suction holes 322, for example spaced around the window 360.

In the depicted embodiment, the housing 312 comprises a hard plastic molding, for example formed of 3.0 mm thick black ABS material. In alternate forms, the shell may comprise other plastics, composites, metals or other materials. FIG. 11 shows an assembly view of the suction device 310. The housing 312 comprises an upper half shell 370 and a lower half shell 380. One or both of the upper and lower half shells 370, 380 define a notch or cutout section 382a, 382b, into which a slotted H-shaped mounting hub 352 of the tubing coupling 350 is engaged. The upper and lower half shells 370, 380 further comprise an inner collar or rim 390, 392 extending generally transversely from the top and bottom panels 330, 320, which bound and define the perimeter of the window 360 when assembled. The inner collar of one of the top and bottom half shells 370, 380 is optionally larger than the inner collar of the other (for example having an inside dimension substantially equal to or slightly larger than a corresponding outside dimension of the other inner collar), so that one collar nests within the other when assembled with a close fit, forming a substantially fluid-tight seal. The upper and lower half shells 370, 380 further comprise an outer collar or peripheral flange 402, 404 extending generally transversely from the top and bottom panels 330, 320, which nestingly engage one another in similar fashion with a close fit, forming a substantially fluid-tight seal, to comprise the front, back and side faces 340, 342, 344, 346.

The suction device 310 is assembled by aligning the slots of the mounting hub 352 of the tubing coupling with the cutout sections 382a, 382b of the upper and lower half shells 370, 380, and engaging the upper and lower half shells to one another with the inner collars 390, 392 and the peripheral flanges 402, 404 aligned and nesting or slidingly engaging together. A friction fit, one or more snap couplings, fasteners, adhesive, thermal or solvent welding or other attachment means are optionally provided to hold the upper and lower half shells 370, 380 together. Optionally, the attachment means is detachable, to allow disassembly and separation of the upper and lower half shells 370, 380 after use, for example to retrieve an object inadvertently sucked into the device 310. Alternatively, the upper and lower half shells 370, 380 are permanently or semi-permanently attached to one another. Engagement of the edges of the peripheral flanges 402, 404 surrounding the cutout sections 382a, 382b of the upper and lower half shells 370, 380 with the slots of the mounting hub 352 retain the tubing coupling 350 captive in place with respect to the housing 312.

In use, the suction device 310 is placed onto a floor or other surface or in an area from which fluid is to be collected, with the bottom panel or suction surface 320 positioned downwardly upon or adjacent the floor or other surface. A suction tube or hose is connected to the tubing coupling 350, for example by friction engagement with the ribbed outer surface of the tubing coupling, and suction is applied from an external suction source. The suction creates a vacuum within the hollow internal chamber formed between the upper and lower half shells 370, 380, drawing airflow and liquids inwardly through the suction holes 322 in the bottom panel or suction surface 320. The inner window 360 creates a vortex or circumferential flow of air and fluids inside the housing 312, and outward through the suction port of the tubing coupling 350. The user may use their foot placed on the top panel 330 or other motive means to move the suction device around on the floor where fluids are to be collected.

The suction device 310 can optionally be used in combination with an absorbent pad, sleeve, sheet or the like, or alternatively can be used alone without an absorbent component. If an absorbent component is utilized, in example forms, a non-woven fabric or other absorbent material is applied to at least the bottom panel or suction surface 320 of the suction device 310 and placed in contact with the floor or other surface from which fluid is to be collected. Optionally, the absorbent component may be replaceable, or alternatively may be permanently attached to the suction device 310.

While the invention has been described with reference to preferred and example embodiments, it will be understood by those skilled in the art that a variety of modifications, additions and deletions are within the scope of the invention, as defined by the following claims.

What is claimed is:

1. A surgical floor suction device comprising a hollow shell defining an internal suction chamber and a suction surface comprising a plurality of suction holes extending therethrough in fluid communication with the internal suction chamber, the floor suction device further comprising a suction coupling extending from the hollow shell and defining a suction conduit extending into fluid communication with the internal suction chamber.

2. The surgical floor suction device of claim 1, wherein the hollow shell comprises a central window extending therethrough, with the internal suction chamber defined around the central window.

3. The surgical floor suction device of claim 1, wherein the hollow shell comprises first and second half-shells having interengaging peripheral flanges.

4. The surgical floor suction device of claim 3, wherein the first and second half-shells comprise moldings formed of a substantially rigid plastic material.

5. The surgical floor suction device of claim 3, wherein one of the first and second half-shells comprise the plurality of suction holes.

6. The surgical floor suction device of claim 5, wherein the plurality of suction holes are arranged around a central aperture extending through the first and second half-shells.

7. The surgical floor suction device of claim 6, wherein the first and second half-shells comprise interengaging inner collars around the central aperture.

8. The surgical floor suction device of claim 3, wherein the first and second half-shells comprise cutout sections for engaging a slotted mounting hub of the suction coupling.

9. The surgical floor suction device of claim 1, further comprising an absorbent component for application to the suction surface.

10. A surgical floor suction device comprising a hollow shell defining a central aperture and an annular internal suction chamber surrounding the central aperture, the shell having a suction surface comprising a plurality of suction holes spaced around the central aperture and extending through the shell into fluid communication with the internal suction chamber.

11. The surgical floor suction of claim 10, further comprising a suction coupling extending from the hollow shell and defining a suction conduit extending into fluid communication with the internal suction chamber.

12. The surgical floor suction device of claim 10, wherein the hollow shell comprises first and second half-shells having interengaging peripheral flanges.

13. The surgical floor suction device of claim 12 wherein the first and second half-shells comprise moldings of a substantially rigid plastic material.

14. The surgical floor suction device of claim 13, wherein one of the first and second half-shells comprise the plurality of suction holes.

15. The surgical floor suction device of claim 12 wherein the first and second half-shells comprise interengaging inner collars around the central aperture.

16. The surgical floor suction device of claim 11, wherein the first and second half-shells comprise cutout sections for engaging a slotted mounting hub of the suction coupling.

17. The surgical floor suction device of claim 10, further comprising an absorbent component for application to the suction surface.

18. A surgical floor suction device comprising a hollow shell defining a central aperture and an annular internal suction chamber surrounding the central aperture, the shell having a suction surface comprising a plurality of suction holes spaced around the central aperture and extending through the shell into fluid communication with the internal suction, and further comprising a suction coupling extending from the hollow shell and defining a suction conduit extending into fluid communication with the internal suction chamber.

19. The surgical floor suction device of claim 18, wherein the hollow shell comprises first and second half-shells having interengaging peripheral flanges.

20. The surgical floor suction device of claim 19, wherein the first and second half-shells comprise moldings formed of a substantially rigid plastic material.

21. The surgical floor suction device of claim 19, wherein the first and second half-shells comprise interengaging inner collars around the central aperture.

22. The surgical floor suction device of claim 19, wherein the first and second half-shells comprise cutout sections in their interengaging peripheral flanges for engaging a slotted mounting hub of the suction coupling.

23. The surgical floor suction device of claim 18, further comprising an absorbent component for application to the suction surface.
